(19) United States
(12) Patent Application Publication
(10) Pub. No.: US 2005/0282238 A1
(43) Pub. Date: Dec. 22, 2005

(54) High-sensitivity chemiluminescent ELISA prion detection method

(76) Inventors: David J. Bowen, Oregon, WI (US); John A. McMaster, Madison, WI (US); Don R. Warren, Stoughton, WI (US)

Correspondence Address: Intellectual Property Department;DEWITT ROSS & STEVENS S.C., US Bank Building, Suite 401, 8000 Excelsior Drive, Madison, WI, 53717-1914, US

(21) Appl. No.: 10/948,469

(22) Filed: Sep. 23, 2004

Related U.S. Application Data
(60) Provisional application No. 60/505,289, filed on Sep. 23, 2003.

(51) Int. Cl. G01N 33/53; G01N 33/537; G01N 33/543

(52) U.S. Cl. 435/7.93

(57) ABSTRACT

A high-sensitivity chemiluminescent ELISA-based prion detection method is provided, the method comprising an ELISA plate coated with monoclonal antibody for the capture of PrPsc protein and treated with an optimized blocking reagent, and a set of labeling and detection reagents. The digestion cocktail is preferably a unique combination of proteolytic, lipolytic and nuclease enzymes for the liberation of the prion PrPsc antigen. The ELISA plate is preferably a multi-well, high-protein binding, microtiter plate coated with an anti-prion monoclonal antibody, used to capture the PrPsc antigen. The blocking agent is an optimized protein and buffer solution. The detection antibody is preferably a biotinylated monoclonal antibody. The chemiluminescent conjugate is preferably a conjugate of streptavidin and alkaline phosphatase enzyme. The final component is preferably an optimized chemiluminescent substrate for maximum sensitivity.

### CROSS-REFERENCE TO RELATED APPLICATION

This application claims priority under 35 U.S.C. §119(e) to U.S. Provisional Application Ser. No. 60/505,289 filed 23 Sep. 2003, the entirety of which is incorporated herein.

### FIELD OF THE INVENTION

The present invention relates generally to methods of prion detection. More specifically, the invention provides a high-sensitivity chemiluminescent ELISA-based method for the detection of the pathogenic form of the prion protein PrPsc in tissues and fluids.

### BACKGROUND OF THE INVENTION

A prion is a normal cellular protein encoded by a single exon of a single copy gene. Prions are found predominantly on the surface of neurons and are attached thereto by a glycoinositol phospholipid anchor. Normal prions are protease sensitive. The normal prion protein PrPc is thought to be involved in the synaptic function of the brain. The abnormal prion protein PrPsc is thought to cause spongiform encephalopathy diseases such as scrapie. The PrPsc protein is relatively resistant to proteases.

Abnormal PrPsc is thought to cause prion diseases known as spongiform encephalopathies. Spongiform encephalopathies are characterized by large, abnormal vacuoles in the cortex and cerebellum of disease-afflicted animals. Spongiform encephalopathy diseases represent a spectrum of diseases affecting both humans and animals. For instance, sheep develop scrapie, mink develop Transmissible Mink Encephalopathy (TME), elk and muledeer develop Chronic Wasting Disease (CWD) and cows develop Bovine Spongiform Encephalopathy (BSE).

In humans, abnormal PrPsc is thought to cause such diseases as Creutzfeld-Jacob Disease (CJD), Gerstmann-Straussler-Scheinker syndrome (GSS), Fatal Familial Insomnia (FFI), Kuru, and Alpers Syndrome. The most common form of human spongiform encephalopathies is sporadic CJD (sCJD). sCJD is a rare, progressive and fatal neurodegenerative disorder with a worldwide incidence of 0.5-2 new cases per year per million population. sCJD is characterized clinically by dementia, ataxia and myoclonus, and histopathologically by astrogliosis, dendritic spongiosis and neuronal loss. In addition to sCJD and hereditary forms, acquired forms of CJD also exist. For example, Kuru results from the consumption of contaminated material, and iatrogenic CJD is acquired through transplantations and medical procedures using contaminated material. Further, a new variant of CJD (vCJD) was observed in humans approximately ten years after the 1986 outbreak of BSE in the United Kingdom. In contrast to patients with sCJD, those with vCJD showed abnormal PrPsc in lymphatic organs such as the tonsils. The appearance of vCJD led to concern regarding the transmission of animal forms of spongiform encephalopathies to humans.

Humans can acquire spongiform encephalopathy diseases by inheriting it or acquiring it. Spongiform encephalopathies are usually acquired through ingesting contaminated material from an afflicted animal. Most acquired cases of spongiform encephalopathies occur by eating material from an afflicted animal, such as uncooked beef from a cow afflicted with Mad-Cow Disease, formally known as Bovine Spongiform Encephalopathy (BSE). However, spongiform encephalopathies have also been acquired through medical procedures using contaminated donor products, such as corneal transplants from a sheep having scrapie. Further, spongiform encephalopathies have been acquired through the use of growth hormone injections containing contaminated material.

Experiments with transgenic mice expressing bovine PrPc show that spongiform encephalopathy diseases may be transmitted to humans. Where mice were inoculated with PrPsc tissue, either from patients with vCJD or sCJD or from cattle affected by BSE, the “signature” of disease was identical in both BSE and vCJD.

These findings led to new uncertainties and intense discussions regarding methods of detecting prion levels in products. Although transmission of spongiform encephalopathies through tissue samples such as whole blood has been demonstrated in animals, the amount of infectious agent needed to transmit disease by an intravenous route to humans makes transmission by therapeutic blood products unlikely. Luckily, no case of transmission of CJD by blood transfusion or administration of blood products in humans has ever been identified. However, continued concern has resulted in increased demand for a rapid assay capable of detecting PrPsc levels in tissue.

Once acquired, spongiform encephalopathy diseases are characterized by loss of motor control, dementia, paralysis, wasting and eventually death. However, details regarding the pathogenesis of spongiform encephalopathy diseases remain largely unknown.

As early as 1967, it was postulated that a self-replicating infectious agent was the cause of spongiform encephalopathies such as scrapie. In 1982 the “prion hypothesis” was proposed, which assumes that the prion protein, a small proteinaceous particle not associated with nucleic acids, is the major and possibly only infectious particle in spongiform encephalopathy diseases.

The cellular function of the prion is unclear. Its capability for binding bivalent metals such as copper have prompted speculation that prions are directly linked to cellular resistance to oxidative stress. Further, a prions superoxide dismutase activity suggests prions may be important for synaptic activity. Superoxide dismutase (SOD) enzymes are the most common way aerobes detoxify superoxide. In humans, a number of pathologies involve the overproduction of superoxide through inflammatory pathways. A large number of animal models of disease have shown that genetically engineered mice which lack SODs are more sensitive to the modeled disease, while mice that over-express SODs are more resistant to the modeled disease.

Experiments have also suggested that normal PrPc may play a role in spongiform encephalopathy diseases. Normal PrPc possess neurotoxic properties and verexpression of normal PrPc in transgenic mice can induce a spongiform-like degeneration of the nervous system. The potential pathogenicity of PrPc adds confusion to our understanding of the role prions play in spongiform encephalopathy diseases.

The infectivity of spongiform encephalopathy diseases is thought to be caused by a structural change of the normal PrPc. Normal PrPc is soluble and is usually found in healthy cells. Spongiform encephalopathy diseases seem to occur when normal PrPc changes into an insoluble form that has a tendency to form fibrils, the abnormal PrPsc. However, diseases can occur with very low levels of PrPsc present in tissue

Therefore, a need exists for a method of detecting even very low levels of PrPsc in tissue. Methods for the detection of prions have been in use for years. Typically, prion detection methods include immunohistochemistry kits, western blot detections, direct-binding Enzyme Linked ImmunoSorbent Assays (ELISA) with colorometric detection methods, and sandwich ELISA with colorometric detection methods. Currently, a definitive diagnosis of spongiform encephalopathy is only possible by post mortem immunohistochemical identification of PrPsc in tissue from afflicted patients.

The most common prion detection method uses proteinase K treatment and Western blotting analysis. Unfortunately, this method is a very sophisticated, time-consuming technique. Further, this method requires highly technical and expensive equipment, meaning that only very specialized laboratories and hospitals can conduct this method. See for example U.S. Pat. No. 6,750,025 to Hammond et al. and U.S. patent application Ser. No. 2002/0137114 to Voelkel et al.

Other prion detection methods use proteins such as S-100 proteins, tau proteins, neuron-specific enolase, creatine kinase, myelin basic protein and 14-3-3 proteins as surrogate markers for spongiform encephalopathy diseases. However, these proteins remain problematic for use in screening assays (especially for CJD) for a variety of reasons: detection levels may be very low, some proteins do not exist in the cerebrospinal fluid, some proteins cross-react with non-brain isoforms, and some protein levels may change in the late stages of disease.

Still other conventional prion detection methods use monoclonal antibodies to measure PrPsc levels in tissue. However, these methods require the antibodies be denatured, thereby limiting our study of the protein. Monoclonal antibodies specific for PrPsc have been reported, but their utility has not been verified. Other monoclonal antibodies have been suggested, but in general the specificity and affinity of these antibodies for PrPsc remains low. The major problem in generating a sufficient immunological response to provide useful antibodies is because the structure of PrPsc is highly conserved in animals. Consequentially, immunogens comprising prions from different species are recognized by the immunized animals as host proteins.

Another problem with conventional prion detection methods is a lack of sensitivity. Distinguishing between normal PrPc and abnormal PrPsc is very important; therefore, any detection method must have a high sensitivity. Further, samples with very low levels of PrPsc may yield false negatives, allowing contamination to spread.

A further problem with conventional prion detection methods is the limited automation potential of these methods. Improving automation improves the speed of diagnosis, saving costs and time. In instances where entire herds of cattle, sheep or deer may require slaughter to eliminate a spongiform encephalopathy disease, the quicker the diagnosis, the better.

Another problem with conventional prion detection methods is the invasive procedures required to procure the necessary samples. Because PrPsc proteins are not clearly understood, non-invasive methods of procuring samples eliminates the chance of harming the protein before testing. Therefore, a need exists for a rapid, automated prion detection method that can quickly and efficiently determine the presence of PrPsc protein in tissue samples obtained using non-invasive procedures.

### SUMMARY OF THE INVENTION

A high-sensitivity chemiluminescent ELISA-based prion detection method and a corresponding kit are provided for the detection of the pathogenic form of the prion protein PrPsc in a tissue sample. The kit comprises an ELISA plate coated with an immobilized monoclonal antibody for the capture of PrPsc, a detection antibody, a chemiluminescent conjugate, and a chemiluminescent substrate. The method compromises contacting a tissue sample with an immobilized monoclonal antibody, whereby an amount of PrPsc is bound to the antibody. The bound PrPsc is then contacted with a labeled detection antibody, thereby yielding an amount of detection-labeled PrPsc. The amount of detection-labeled PrPsc is then contacted with a chemiluminescent conjugate, thereby yielding an immobilized conjugate. The immobilized conjugate is then contacted with a commercial chemiluminescent substrate, thereby generating a chemiluminescent signal. In this manner, the presence of PrPsc in a sample is detected.

The sample to be tested for the presence of PrPsc is preferably tissue from human, bovine or sheep brain. Tissue from lymphatic organs may also be used. The sample is digested using a digestion cocktail comprising a combination of proteolytic, lipolytic and nuclease enzymes for the liberation of PrPSc. Once digested, the sample is added to an ELISA plate. The ELISA plate is a multi-well, high-protein binding, microtiter plate coated with an immobilized anti-prion monoclonal antibody. The anti-prion monoclonal antibody is used to capture the PrPSc from the sample.

The immobilized monoclonal antibody used to capture the PrPsc in the sample and bind it to the plate is preferably comprised of an optimized protein and buffer solution. The labeled detection antibody is preferably a biotinylated monoclonal antibody, including but not limited to antibodies 99/97.6.1, 89/193.1.5, and 86/160.5.1. The chemiluminescent conjugate used to contact the detection-labeled PrPsc in the sample is preferably a conjugate of streptavidin and alkaline phosphatase enzyme. The chemiluminescent substrate contacts the immobilized conjugate to generate a chemiluminescent signal. The chemiluminescent signal is measured using any method known to the art.

A primary object of the present invention is to provide a simple, easy-to-use, high-sensitivity chemiluminescent ELISA-based prion detection method that will overcome the shortcomings of the prior art methods.

A further object of the present invention is to provide a high-sensitivity chemiluminescent ELISA-based prion detection method for the detection of PrPsc in a tissue sample.

A further object of the present invention is to provide a high-sensitivity chemiluminescent ELISA-based prion detection method that is faster than conventional methods.

Another object of the present invention is to provide a high-sensitivity chemiluminescent ELISA-based prion detection method for the detection of very low levels of PrPsc in a sample.

Another object of the present invention is to provide a high-sensitivity chemiluminescent ELISA-based prion detection method that has a heightened automation potential for high-throughput screening.

Another object is to provide a high-sensitivity chemiluminescent ELISA-based prion detection method that allows for the detection of PrPSc in specimens obtained using noninvasive procedures.

The objects and advantages of the invention will appear more fully from the following detailed description of the preferred embodiment of the invention made in conjunction with the accompanying drawings.

### BRIEF DESCRIPTION OF THE FIGURES

FIG. 1 is a flowchart that describes each of the steps in the test method.

FIG. 2 is a drawing of events that occur in each well on the test ELISA plate.

FIG. 3 is a graph showing the results of four different types of chemiluminescent reagents.

### DETAILED DESCRIPTION OF THE INVENTION

Turning now descriptively to the figures, the attached figures illustrate a high-sensitivity chemiluminescent ELISA-based prion detection method. The method disclosed for the detection of PrPsc in a biological specimen is an ELISA of the type generically referred to as a “sandwich ELISA.” Here, PrPsc is sandwiched between an immobilized capture antibody and a detection antibody.

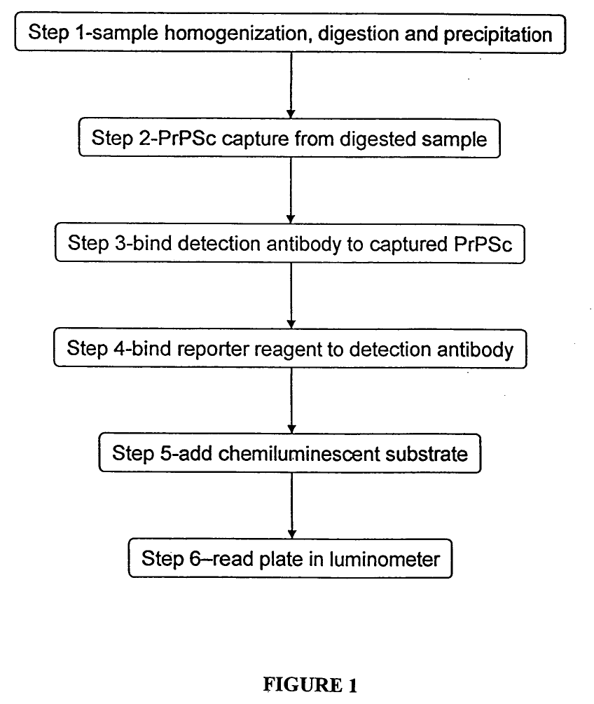

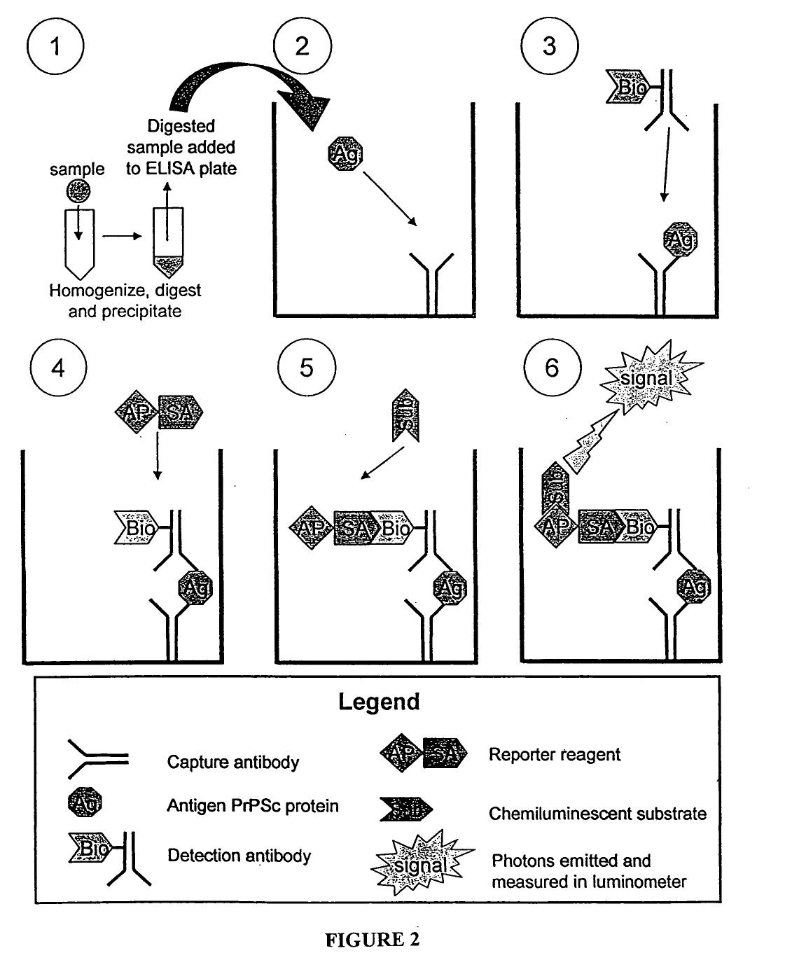

FIGS. 1 and 2 show a flowchart and detailed description of the method of the present invention. The method comprises six basic steps. In step one, the sample is homogenized and digested with a unique cocktail of undigested enzymes. The digestion enzymes liberate the PrPsc protein from the other material in the sample, allowing the PrPsc protein to be precipitated out.

The digestion cocktail utilized in the present invention is a unique combination of proteolytic, lipolytic and nuclease enzymes designed for the liberation of PrPsc from the sample. The digestion cocktail consists of pancreatin, proteinase K, lipase and nuclease supplied in an optimized mixture to solubilize the sample. Further, digestion eliminates normal PrPc from the sample without impacting the abnormal PrPsc in the sample. While in the present invention the cocktail has been optimized for the digestion of human, bovine or sheep brain, it is envisioned that the digestion cocktail may be further optimized for other species.

Further, it is within the scope of the present invention to use this prion detection method in combination with other anti-prion ligands and detection antibodies. One skilled in the art will understand that depending on the choice of antibody, the concentration of pancreatin, protease, lipase and nuclease in the cocktail may need to be adjusted. Further, conditions such as incubation time and temperature of the digestion step may be altered to assure the maximum PrPsc presence while eliminating any PrPc from the sample.

In step two, the precipitated PrPsc protein is dissolved in a buffer and added to the wells of a fully-blocked ELISA plate. The ELISA plate is preferably a multi-well, high-protein binding, microtiter plate coated with anti-prion ligand specific for PrPsc. The ELISA plate utilized in the present invention preferably consists of a multi-well microtiter plate that is opaque to eliminate “cross-talk.” Cross-talk is light that comes from wells adjacent to the well that is being read.

The ELISA plate can be of any type known in the art including but not limited to a Wallac BW Isoplate HB having a high-protein binding capacity, a Xenopore Xenobind White, a Nalge Nunc Flatbottom Immunowhite Maxisorb plate having a high glycoprotein binding capacity, or a Paul Life Sciences Acrowall filter plate with a 0.2 micrometer biotrace NT membrane having a high-protein binding capacity.

Regardless of which type of plate is chosen, the ELISA plate is preferably coated with an immobilized anti-prion ligand specific for PrPsc. This anti-prion ligand is used to capture the PrPsc present in the sample. An anti-prion ligand refers to a monoclonal or polyclonal antibody, a peptide, phage, protein, DNA or RNA or other non-biological polymers which specifically recognizes PrPsc. While in the preferred embodiment of the present invention a monoclonal antibody is used as the anti-prion ligand, other ligands may be used.

Between each of steps two through five, the ELISA plate is washed twelve times with Phosphate-Buffered Saline (PBS) containing Tween 20 in an automatic plate washer.

In step three, a blocking agent is applied to the wells of the ELISA plate. Most often, blocking agents comprise a solution of protein and polyoxyethylenesorbitan monolaurate (Tween 20). The blocking agent binds to sites on the ELISA plate that are not already bound to captured PrPsc. The unoccupied binding sites must be fully blocked by the blocking agent to prevent the nonspecific binding of agents added in subsequent steps. The blocking agent may also be added to the ELISA wells in combination with the digested sample.

In the preferred embodiment of the present invention, the blocking agent consists of an optimized mixture of high-purity casein, Tween 20 and PBS (pH 7.2). Possible variations of the blocking agent include the use of gelatin, bovine serum albumin, non-fat dry powdered milk or any optimized protein in a buffer that does not interfere with the method of the present invention.

In step four, a labeled detection antibody is added to the wells of the ELISA plate. This labeled antibody binds to the captured PrPsc in the wells, creating an amount of detection-labeled PrPsc. The detection antibody preferably consists of an optimized protein and buffer solution. The detection reagent is preferably a biotinylated monoclonal antibody including but not limited to antibodies 99/97.6.1, 89/193.1.5, and 89/160.5.1. Biotinylated refers to a biotin moiety covalently attached to a protein or peptide for the purpose of reacting with another substance such as alkaline or streptavidin in a detection assay.

In step five, the ELISA plate is contacted with a chemiluminescent conjugate. The purpose of the chemiluminescent conjugate is to bind to the detection-labeled PrPsc and create an immobilized conjugate. The chemiluminescent conjugate used in the present invention is preferably a conjugate of streptavidin and alkaline phosphatase. However, other reagents such as streptavidin and horseradish peroxidase conjugates and streptavidin combined with calf intestinal alkaline phosphatase may also be used. When a chemiluminescent substrate is added, the conjugate provides the enzymatic activity necessary to produce a light emission. Upon contact with the chemiluminescent substrate, the immobilized chemiluminescent conjugate generates a chemiluminescent signal.

Chemiluminescent substrates are a widely used and convenient means for measuring the effects of experimental conditions on cellular physiology. For instance, Promega's Luciferase Assay of Madison, Wis., uses light produced by converting the chemical energy of luciferin oxidation through an electron transition, forming the product molecule oxyluciferin. Common chemiluminescent substrates include, but are not limited to, such substrates as CSPD/Sapphire, CSPD/Emerald, CDP/Sapphire or CDP/Emerald (Applied Biosystems, Bedford, Mass.). For best results, all chemiluminescent substrates should be used according to the manufacturer's instructions.

In step six, the chemiluminescent signal generated by the chemiluminescent substrate contacting the immobilized conjugate is measured. The chemiluminescent signal may be measured by any means known to the art. For instance, in a preferred embodiment of the present invention, the ELISA well containing the chemiluminescent signal may be read in a microtiter plate luminometer. A luminometer measures the amount of light emitted from a well that contains the captured, detection-labeled PrPSc. The amount of light produced in the well is directly proportional to the amount of detection-labeled PrPsc in the well.

The present invention also includes a test kit. There are five main components of the test kit designed to detect the presence of the pathogenic prion, PrPSc. In a preferred embodiment, the kit comprises an ELISA plate coated with an immobilized monoclonal antibody, a labeled detection antibody, a chemiluminescent conjugate and a chemiluminescent substrate. The ELISA plate is preferably coated with immobilized monoclonal antibody 89/193.1.5. The labeled detection antibody is preferably a biotinylated form of the monoclonal antibody 99/97.6.1. The chemiluminescent conjugate is preferably streptavidin and alkaline phosphatase and the chemiluminescent substrate is preferably Sapphire II.

However, in an alternate embodiment, monoclonal antibody 89/160.5.1 is used as the capture antibody and the biotinylated form of the antibody 99/97.6.1 as the labeled detection antibody. Streptavidin and horse radish peroxidase constitute the chemiluminescent conjugate and CDP/Emerald is the chemiluminescent substrate.

In yet another alternate embodiment of the present invention, monoclonal antibody 99/97.6.1 is used as the capture antibody and a biotinylated form of monoclonal antibody 89/160.5.1 acts as the labeled detection antibody. Monoclonal antibody 99/97.6.1 may also be used as the capture antibody and a biotinylated form of monoclonal antibody 89/193.1.5 may be used as the labeled detection antibody.

It is to be realized that the optimum relationship for the components of the invention as described above is to include variations in ELISA plates, capture antibodies, detection antibodies, chemiluminescent substrates, configuration and use, are all deemed readily apparent and obvious to one skilled in the art. Further, all equivalent configurations to those illustrated in the drawings and described in the specification are intended to be encompassed by the present invention.

The chemiluminescent assay of the present invention has been used to detect both recombinant prion protein that was produced in bacteria and the scrapie form of the prion protein from infected sheep brain, as seen from the examples described herein.

### EXAMPLE ONE

Recombinant Prion Protein

A 99% pure protein solution containing 1 μg protein/1 ml blocking buffer was used as the starting sample material for the assay. In alternate embodiments, this sample may be further diluted before being added to the 96 well plate.

The blocking buffer used was Phosphate-Buffered Saline (PBS) (pH 7.4), having 0.05% Tween 20 detergent. One of the following proteins was then added in the amounts noted: Bovine serum albumin (5%), Gelatin (1.5%), non-fat powdered milk (5%), or highly purified casein (0.2%).

The 96 well assay plates were then coated with capture antibody 89/193.1.5 by adding 2 μg to 10 μg of the monoclonal antibody in PBS (pH 7.4).

Next, 100 μl of the protein sample diluted in blocking buffer was added to each well in a 96 well plate. The plate was then incubated at room temperature for 1 hour.

Next, each plate was washed 12 times with PBS+0.05% Tween 20 using an automatic plate washer. After washing, 100 μl of a solution containing a biotin labeled detection antibody in the blocking buffer was added to the wells and incubated for 1 hour at room temperature.

Each plate was then washed 12 times with PBS and 0.05% Tween 20 using an automatic plate washer. After washing, 100 μl of a strepavidin-alkaline phosphatase conjugate solution was added to the wells and incubated for 1 hour at room temperature.

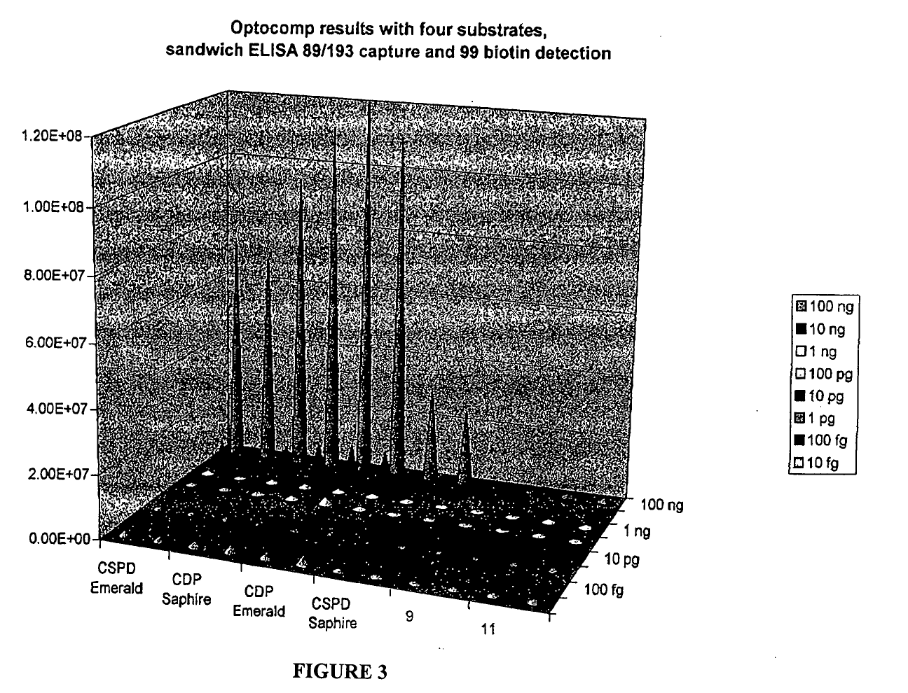

Finally, each plate was washed 12 times with PBS and 0.05% Tween 20 using an automatic plate washer. The plate was washed with an alkaline phosphatase assay buffer containing 20 mM Tris (pH 9.8) and 10 mg MgCl. CDP Star Emerald II (Applied Biosystems, Bedford, Mass.) was added to the wells and light emission was recorded using an Optocomp II Luminometer (MGM Instruments, Hamden Conn.). The amount of light generated reflected the amount of the recombinant prion protein in the sample (See FIG. 3).

### EXAMPLE TWO

Prion Protein From Sheep Brain

Sheep brain material was prepared using Phosphotungstic Acid Precipitation (PTA). First, tissue samples of sheep brain ranging in size from 0.2-0.25 grams were prepared. The tissue was minced into small pieces with a clean scalpel and placed into a 2-ml microcentrifuge tube containing beads (1.0 mm Zirconia beads, BioSpec Products, Inc., Product Number 11079110 zx) and 1 ml of direct lysis buffer. The sample was ground together using a Fast Prep Machine at speed 6 for 45 sec. The mixture was then incubated at room temperature for at least 30 min. After incubation, the tissue sample can be tested or stored at −20° C.

When ready for testing, the tissue sample was removed from storage in a −20° C. freezer and 600 μl of the prepared tissue sample was added to an equal volume of PBS containing 4% Sarkosyl. The mixture was vortexed well and incubated at 37° C. for 10 min. (The pipette tip should be cut off the point for easy transfer of the tissue sample).

After vortexing and incubation, 100 μg/ml of Dnase was added to the tissue sample. The Dnase and tissue sample were mixed well and incubated at 37° C. for 30 min. After an additional brief vortex and spin at 4000 rpm for 5 min., 1 ml of the supernatant was transferred to a screw-top 2 ml tube (Sarstedt, Product Number 72694006).

Proteinase K was then added to the supernatant to achieve a 50 μg/ml final concentration. This solution was vortexed and incubated at 50° C. for 30 min.

After vortexing and incubation, 80 μl of 4% Sodium Phosphotungstic Acid was added to 170 mM MgCl₂ to create a solution having a final concentration of 0.3%. This solution was then vortexed and incubated 37° C. for 40 min. The mixture was then spinned at 14,000 rpm for 30 min before discarding the supernatant and removing any remaining liquid from the tube with absorbent paper, leaving only a small pellet containing PrPsc. (This pellet was usually very small).

In preparing the tissue sample for testing, a lysis buffer was created using 10 mM Trise-HCl buffer (pH 7.5), 0.5% NP-40, 0.5% sodium deoxycholate, 2.4% (w/v) PTA, 170 mM MgCl (pH 7.4), sodium PhosphoTungstic Acid (Sigma Chemical, Product Number p-6395) and 3.4% sarkosyl in PBS (pH 7.4).

Once the sample pellet containing the PrPSc protein was isolated, the pellet was dissolved in 125 mM of Tris-HCl buffer (pH 6.8) containing 8% beta-mercaptoethanol and 5% glycerol. The resulting solution was then diluted 1 to 10 with water (non-denatured) or 1 to 10 with a denaturing buffer containing 65 mM Tris-HCl buffer (pH 6.8) containing 4% beta-mercaptoethanol, 2.5% glycerol and 4 M urea (denatured). The resulting solution was then boiled for 10 minutes.

After the solution containing the diluted pellet cooled to room temperature, the solution was diluted 1/100 (shown as 1 on the chart), 1/1000 (0.1 on the chart), 1/10,000 (0.01 on the chart) and 100,000 (0.001 on the chart) in blocking buffer with 0.2% high purity casein.

Upon dilution, 100 μl of the solution were added to each well in a 96 well plate coated with a capture antibody. The plate was then incubated at room temperature for 1 hour.

After incubation, the plate was washed 12 times with PBS and 0.05% Tween 20 using an automatic plate washer. After washing, 100 μl of a solution containing a biotin-labeled detection antibody in the blocking buffer (with 0.2% high purity casein) was added to the wells and incubated 1 hour at room temperature. In one embodiment, the blocking agent was Bovine Serum Albumin (BSA).

After incubation, each plate was again washed 12 times with PBS+0.05% Tween 20 using an automatic plate washer. After washing, 100 μl of a solution containing a strepavidin-alkaline phosphatase conjugate in the blocking buffer (with 0.2% high purity casein) was added to the wells. The plate was then incubated 1 hour at room temperature.

After incubation, each plate was washed 12 times with PBS+0.05% Tween 20 using an automatic plate washer. The plate was washed with an alkaline phosphatase assay buffer containing 20 mM Tris (pH 9.8) and 10 mg MgCl. A chemiluminescent substrate (CDP Star Emerald II, Applied Biosystems, Bedford, Mass.) was then added to the wells and the resulting light emission was recorded using an Optocomp II Luminometer (MGM Instruments, Hamden Conn.). The amount of light generated reflects the amount of the PrP-sc in the sample.

1. A method of detecting presence of pathogenic prion protein (PrPsc) in a sample, the method comprising:
a) contacting the sample with an immobilized capture antibody specific for PrPsc, whereby an amount of PrPsc is bound to the antibody;
b) contacting the PrPsc bound to the antibody with a labeled detection antibody, thereby yielding an amount of detection-labeled PrPsc;
c) contacting the amount of detection-labeled PrPsc of step (b) with a chemiluminescent conjugate specific for the detection-labeled PrPsc, thereby yielding an immobilized conjugate;
d) contacting the immobilized conjugate with a chemiluminescent substrate, whereby a chemiluminescent signal is generated; and
e) measuring the chemiluminescent signal generated, thereby detecting the presence of detection-labeled PrPsc in the sample.

2. The method of claim 1, wherein the sample is human, bovine or sheep lymph or brain.

3. The method of claim 1, wherein in step (b), the labeled detection antibody is a biotinylated monoclonal antibody.

4. The method of claim 3, wherein the biotinylated monoclonal antibody is 99/97.6.1.

5. The method of claim 3, wherein the biotinylated monoclonal antibody is 89/193.1.5.

6. The method of claim 3, wherein the biotinylated monoclonal antibody is 89/160.5.1.

7. The method of claim 1, wherein in step (c) the chemiluminescent conjugate is a conjugate of streptavidin and alkaline phosphatase.

8. The method of claim 1, wherein in step (c) the chemiluminescent conjugate is a conjugate of streptavidin and calf intestinal alkaline phosphatase.

9. The method of claim 1, wherein in step (c) the chemiluminescent conjugate is a conjugate of streptavidin and horseradish peroxidase.

10. A method of detecting concentration of pathogenic prion protein (PrPsc) in a sample, the method comprising:
a) contacting the sample with an immobilized antibody specific for PrPsc, whereby an amount of PrPsc is bound to the antibody;
b) contacting the PrPsc bound to the antibody with a labeled detection antibody, thereby yielding an amount of detection-labeled PrPsc;
c) contacting the amount of detection-labeled PrPsc of step (b) with a chemiluminescent conjugate specific for the detection-labeled PrPsc, thereby yielding an immobilized conjugate;
d) contacting the immobilized conjugate with a chemiluminescent substrate, whereby a chemiluminescent signal is generated; and
e) measuring the chemiluminescent signal generated, thereby detecting the concentration of detection-labeled PrPsc in the sample.

11. The method of claim 10, wherein the sample is human, bovine or sheep lymph or brain.

12. The method of claim 10, wherein in step (b) the labeled detection antibody is a biotinylated monoclonal antibody.

13. The method of claim 12, wherein the biotinylated monoclonal antibody is 99/97.6.1.

14. The method of claim 12, wherein the biotinylated monoclonal antibody is 89/193.1.5.

15. The method of claim 12, wherein the biotinylated monoclonal antibody is 89/160.5.1.

16. The method of claim 10, wherein in step (c) the chemiluminescent conjugate is a conjugate of streptavidin and alkaline phosphatase.

17. The method of claim 10, wherein in step (c) the chemiluminescent conjugate is a conjugate of streptavidin and calf intestinal alkaline phosphatase.

18. The method of claim 10, wherein in step (c) the chemiluminescent conjugate is a conjugate of streptavidin and horseradish peroxidase.

19. A kit for detecting pathogenic prion protein PrPsc in a sample, the kit comprising, in combination:
a microwell plate having immobilized thereon a capture antibody specific for PrPsc;
a labeled detection antibody specific for PrPsc disposed in a first container;
a chemiluminescent conjugate specific for the detection-antibody and disposed in a second container;
a chemiluminescent substrate specific for the chemiluminescent conjugate; and
instructions for use.

20. The kit of claim 19, wherein the detection antibody is a biotinylated monoclonal antibody.

21. The kit of claim 20, wherein the biotinylated monoclonal antibody is 99/97.6.1.

22. The kit of claim 20, wherein the biotinylated monoclonal antibody is 89/193.1.5.

23. The kit of claim 20, wherein the biotinylated monoclonal antibody is 89/160.5.1.

24. The kit of claim 19, wherein the chemiluminescent conjugate is a conjugate of strepavidin and alkaline phosphatase.

25. The kit of claim 19, wherein the chemiluminescent conjugate is a conjugate of streptavidin and calf intestinal alkaline phosphatase.

26. The kit of claim 19, wherein the chemiluminescent conjugate is a conjugate of streptavidin and horseradish peroxidase.
